(19) United States
(12) Patent Application Publication
(10) Pub. No.: US 2016/0256231 A1
(43) Pub. Date: Sep. 8, 2016

(54) MASTER MANIPULATOR

(71) Applicant: OLYMPUS CORPORATION, Tokyo (JP)

(72) Inventor: Ryohei OGAWA, Tokyo (JP)

(73) Assignee: OLYMPUS CORPORATION, Tokyo (JP)

(21) Appl. No.: 15/159,126

(22) Filed: May 19, 2016

Related U.S. Application Data
(63) Continuation of application No. 14/022,417, filed on Sep. 10, 2013.
Application No. 14/022,417 is a continuation of application No. PCT/JP2012/059128, filed on Mar. 28, 2012.

(30) Foreign Application Priority Data
Mar. 31, 2011 (JP) 2011-079055

(51) Int. Cl. A61B 34/37; A61B 34/00; A61B 46/10; B25J 3/00; G05G 1/02

(52) U.S. Cl. CPC A61B 34/37; B25J 3/00; G05G 1/02; A61B 46/10; A61B 34/74

(57) ABSTRACT

A master manipulator for operating a driving of a slave manipulator includes a grip portion that is positioned in a clean area, gripped by an operator, and provided with a predetermined operating member; and an arm portion that is positioned in an unclean area, and with which the grip portion is directly or indirectly connected. The grip portion has a movable member that moves in conjunction with displacement of the operating member and the arm portion has a position detection portion that detects the position of the movable member.

### FIELD OF THE INVENTION

The present invention relates to a master manipulator that is used for example in a manipulator system or the like. This application is a continuation application of U.S. patent application Ser. No. 14/022,417, filed on Sep. 10, 2013, which is a continuation application based on PCT Patent Application No. PCT/JP2012/059128, filed on Mar. 28, 2012, claiming priority based on Japanese Patent Application No. 2011-079055, filed Mar. 31, 2011, the contents of each of which are incorporated herein by reference.

### BACKGROUND OF THE INVENTION

### Description of Related Art

In recent years, various proposals have been made regarding technology relating to medical procedures using robots. Especially in the field of surgery, various proposals have been made regarding medical manipulator systems that perform procedures on patients with a multi-degree-of-freedom manipulator that has a multi-degree-of-freedom arm.

In a surgical operation that uses a medical manipulator system, the operator operates a master manipulator to control the operation of a slave manipulator (surgical operation instrument) at a location remote from the patient. Generally, since the master manipulator includes electronic components such as a motor and encoder, it is difficult to sterilize the master manipulator.

However, during emergencies in a surgical operation, situations arise in which it is preferable for the operator to perform a procedure by directly contacting the patient. Accordingly, the grip portion that the operator grasps (hereinbelow referred to as the “master grip”) is preferably constituted to be sterilizable. In view of such circumstances, the following art is proposed for example in Japanese Unexamined Patent Application First Publication No. H08-280697.

That is to say, in the art that is disclosed in Japanese Unexamined Patent Application First Publication No. H08-280697, the master grip that the operator grips among the master manipulator is constituted so as to be removable from the master manipulator main body. In the constitution disclosed in Japanese Unexamined Patent Application First Publication No. H08-280697, it is possible to separate the master grip from the master manipulator main body and sterilize it. In other words, the master grip is categorized as a so-called “clean area” member.

Note that a “clean area” refers to a region that can be sterilized or a region that is sterilized. On the other hand, a region that is difficult to sterilize or a region that is not sterilized is called an “unclean area”.

A switch (electrical connection device) for the operator to switch the control method or the like is arranged in a master console of the medical manipulator system. Here, in order to enable the operator to operate the switch without removing his hand from the master grip during operation, it is necessary to have the switch be built into the master grip.

Presently, technology that is constituted so as to be capable of easily sterilizing a site where sterilization treatment is desired in a master manipulator, such as a master grip, is desired.

### SUMMARY OF THE INVENTION

According to a first aspect of the present invention, a master manipulator for operating the driving of a slave manipulator, includes a grip portion that is positioned in a clean area, configured to be gripped by an operator, and provided with a predetermined operating member; and an arm portion that is positioned in an unclean area, and with which the grip portion is directly or indirectly connected. The grip portion has a movable member that moves in conjunction with displacement of the operating member, and the arm portion has a position detection portion that detects the position of the movable member.

According to a second aspect of the present invention, in the master manipulator according to the first aspect, one end portion of the movable member in the longitudinal direction may be inserted into the interior of the arm portion that is connected to the grip portion. The position detection portion detects the position of the movable member, when the movable member is inserted in the interior of the arm portion.

According to a third aspect of the present invention, in the master manipulator according to the first aspect, the grip portion may be provided with a hollow space that houses the movable member, the movable member may be a member that moves in the hollow space of the grip portion in conjunction with displacement of the operating member, and the position detecting portion may detect the position of the movable member in the interior of the hollow space of the grip portion.

According to a fourth aspect of the present invention, in the master manipulator according to any one of the first to third aspects, the movable member may be a member that advances and retracts in the longitudinal direction in conjunction with a linear movement of the operating member, and the position detection portion may detect linear displacement of the movable member.

According to a fifth aspect of the present invention, in the master manipulator according to any one of the first to third aspects, the movable member may be a shaft member that turns in conjunction with a linear movement of the operating member, and the position detection portion may detect rotational displacement of the movable member.

According to a sixth aspect of the present invention, in the master manipulator according to any one of the first to third aspects, the movable member may be a member that advances and retracts in the longitudinal direction in conjunction with a rotating or turning movement of the operating member, and the position detection portion may detect linear displacement of the movable member.

According to a seventh aspect of the present invention, in the master manipulator according to any one of the first to third aspects, the movable member may be a shaft member that turns in conjunction with a rotating or turning movement of the operating member, and the position detection portion may detect turning displacement of the movable member.

According to an eighth aspect of the present invention, the master manipulator according to the second aspect may include an intermediate member where a through hole into which the movable member is inserted so as to freely move is formed. In addition, the grip portion and the arm portion may be connected via the intermediate member, and the movable member may be constituted to be separable at a first portion positioned in the grip, a second portion positioned in the interior of the intermediate member, and a third portion positioned in the interior of the arm portion.

According to a ninth aspect of the present invention, the master manipulator according to any one of the first to third and fifth aspects may include a displacement enlargement mechanism that enlarges and transmits the displacement of the operating member, and the position detection portion may detect the position of the movable member that moves in conjunction with the displacement that is enlarged and transmitted by the displacement enlargement mechanism.

### BRIEF DESCRIPTION OF DRAWINGS

FIG. 1 is a side cross-sectional view that shows one configuration example of the master manipulator according to the first embodiment of the present invention.

FIG. 2 is a side cross-sectional view that shows the vicinity of the switch before the push-down operation of the master manipulator according to the first embodiment of the present invention.

FIG. 3 is a side cross-sectional view that shows the vicinity of the switch after the push-down operation of the master manipulator according to the first embodiment of the present invention.

FIG. 4 is a side cross-sectional view that shows one configuration example of the master manipulator according to the second embodiment of the present invention.

FIG. 5 is a side cross-sectional view that shows the disassembled state of one configuration example of the master manipulator according to the second embodiment of the present invention.

FIG. 6 is a drawing that shows one configuration example of the intermediate member of the master manipulator according to the second embodiment of the present invention.

FIG. 7 is a side cross-sectional view that shows one configuration example of the master manipulator according to the third embodiment of the present invention.

FIG. 8 is a side cross-sectional view that shows one configuration example of the master manipulator according to the fourth embodiment of the present invention.

FIG. 9 is a drawing that shows the master manipulator according to the fourth embodiment of the present invention viewed from the direction shown by the arrow d5 in FIG. 8.

FIG. 10 is a drawing shows one configuration example of the portion of the displacement enlarging mechanism of the master manipulator according to the fifth embodiment of the present invention.

FIG. 11A is a top view that shows one configuration example of the switch of the master manipulator according to the sixth embodiment of the present invention, in the state of the top cover being removed.

FIG. 11B is a side cross-sectional view of the switch of the master manipulator according to the sixth embodiment of the present invention in the case of being constituted so as to cause the moving member to turn.

FIG. 11C is a cross-sectional view along 11C-11C in FIG. 11B of the switch of the master manipulator according to the sixth embodiment of the present invention in the case of being constituted so as to cause the moving member to turn.

FIG. 12 is a side cross-sectional view that shows one configuration example of the master manipulator according to the seventh embodiment of the present invention.

FIG. 13 is a drawing that shows the vicinity of the switch in the case of viewing the master manipulator according to the seventh embodiment of the present invention from the direction indicated by the arrow d9 in FIG. 12.

FIG. 14 is a side cross-sectional view that shows one configuration example of the master manipulator according to the eighth embodiment of the present invention.

FIG. 15 is a drawing that shows the vicinity of the switch of the grip of the master manipulator according to the ninth embodiment of the present invention, viewed from the direction indicated by the arrow d9 in FIG. 12.

FIG. 16 is a drawing that shows the vicinity of the switch of the grip of the master manipulator according to the tenth embodiment of the present invention, viewed from the direction indicated by the arrow d9 in FIG. 12.

FIG. 17 is a drawing that shows the vicinity of the switch of the grip of the master manipulator according to the tenth embodiment of the present invention, viewed from the direction indicated by the arrow d9 in FIG. 12.

FIG. 18 is a side cross-sectional view that shows the vicinity of the switch of the grip of the master manipulator according to the eleventh embodiment of the present invention.

FIG. 19 is a top view of the switch vicinity of the grip of the master manipulator according to the eleventh embodiment of the present invention, viewed from the direction indicated by the arrow A in FIG. 18.

FIG. 20 is a view of the switch vicinity of the grip of the master manipulator according to the eleventh embodiment of the present invention, viewed from the direction indicated by the arrow B in FIG. 18.

FIG. 21 is a view of the switch vicinity of the grip of the master manipulator according to the eleventh embodiment of the present invention, viewed from the direction indicated by the arrow C in FIG. 18.

FIG. 22 is a side cross-sectional view that shows one configuration example of the master manipulator according to the twelfth embodiment of the present invention.

FIG. 23 is a front view shows the scale of the moving member of the master manipulator according to the twelfth embodiment of the present invention.

### DETAILED DESCRIPTION OF THE INVENTION

Hereinbelow, embodiments of the present invention shall be described with reference to the drawings.

### First Embodiment

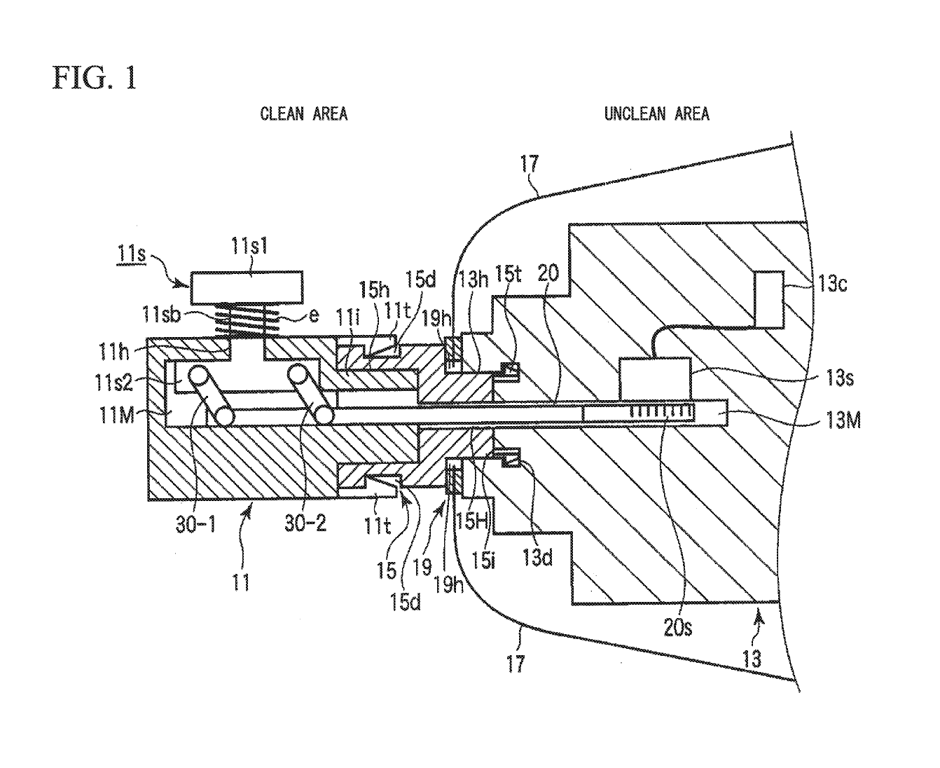

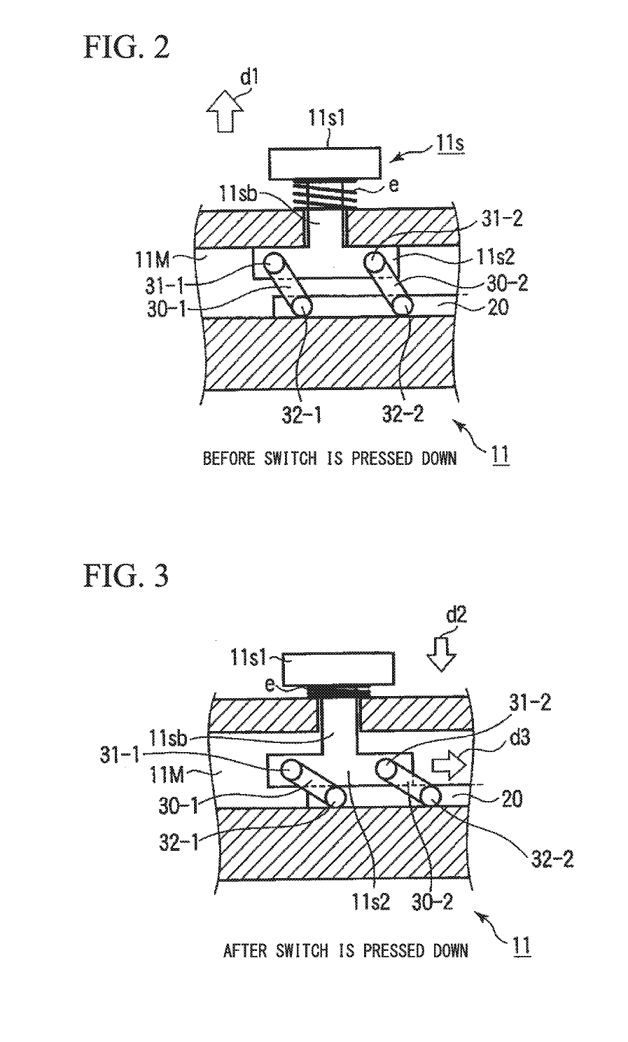

FIG. 1 is a side cross-sectional view that shows one configuration example of the master manipulator according to a first embodiment of the present invention. FIG. 2 is a side cross-sectional view that shows the vicinity of the switch before the push-down operation of the master manipulator according to the first embodiment of the present invention. FIG. 3 is a side cross-sectional view that shows the vicinity of the switch after the push-down operation of the master manipulator according to the first embodiment of the present invention. Note that since FIG. 1 to FIG. 3 are side cross-sectional views, and the outer shapes are not shown. However, the outer shape of the master manipulator according to the first embodiment assumes a columnar shape as a whole, in the same way as an ordinary master manipulator. The columnar shape is, for example, a cylindrical shape or a polygonal columnar shape. Also, although not illustrated, the shape of the master grip may be that of a surgical tool shape of forceps, scissors, and a scalpel or the like as used in an ordinary surgical operation by a medical doctor.

As shown in FIG. 1, the master manipulator according to the first embodiment includes a grip 11 (a grip portion) that is grasped by the operator, a master arm main body 13 (an arm portion), an intermediate member 15, a drape 17, a drape connector 19, and a movable member 20.

The grip 11 has an insertion projection portion 11i, a hollow region 11M, a switch 11s, and an engagement projection portion 11t.

The grip 11 is a member that has a hollow structure (hollow space) with an outer cylindrical shape in which the insertion projection portion 11i is provided. The insertion projection portion 11i of the grip 11 is inserted into an insertion hole 15h of the intermediate member 15. A hollow region 11M that is a hollow space is formed in this grip 11. This hollow region 11M is opened to external space on the insertion projection portion 11i side. A through hole 11h for attaching the switch 11s described below is formed as a hole portion that penetrates from the side periphery of the grip 11 to the hollow region 11M.

The switch 11s is a member having an approximately H shape in side cross section as shown from FIG. 1 to FIG. 3. The switch 11s has a first member 11s1, a second member 11s2, and a bar member 11sb. The first member 11s1 is positioned outside of the hollow region 11M. The second member 11s2 is housed inside the hollow region 11M of the grip 11. The bar member 11sb is inserted into the through hole 11h of the grip 11, and connects the first member 11s1 and the second member 11s2.

In addition, the shape of this switch 11s is not limited to the aforementioned example, but it may be arbitrarily configured as long as it is provided with a shape that functions as a switch (for example, it may utilize a shape that constitutes a well-known switch).

Here, a resilient member e is provided between the first member 11s1 and the side periphery of the grip 11. The resilient member e presses the first member 11s1 toward the outside in the diameter direction of the grip 11 (the direction shown in FIG. 2 by the arrow d1). Specifically, this resilient member e is a spring into which the bar member 11sb is inserted.

In the ordinary state, that is, in the state in which an external force is not applied, the first member 11s of the switch 11s is pushed to the outside in the diameter direction of the grip 11 due to the repulsive force of the resilient member e. And in the hollow region 11M of the grip 11, the second member 11s2 is pressed against the inner wall surface of the hollow region 11M of the grip 11.

The movable member 20 is attached to the second member 11s2 of the switch 11s by two link members 30-1 and 30-2 that have the same length. The movable member 20 is approximately bar shaped, and the longitudinal direction of the master manipulator is made its longitudinal direction.

The second member 11s2 and the movable member 20 constitute a well-known parallel link mechanism with four joints by these two link members 30-1 and 30-2.

Here, turning shafts 31-1 and 31-2 are mounted on the side face of a second member 11s2, and turning shafts 32-1 and 32-2 are mounted on the side face in the movable member 20. Here, the distance between the turning shaft 31-1 and the turning shaft 31-2 is the same as the distance between the turning shaft 32-1 and the turning shaft 32-2.

Moreover, as shown in FIG. 2, in the state where the switch 11s is not pushed down, the positions of the turning shafts 31-1 and 31-2 are respectively somewhat more to the operator's side, that is, the left side in FIG. 2, than the positions of the turning shafts 32-1, 32-2 of the movable member 20.

The link member 30-1 has a shaft hole on the end portion of the second member 11s2 side, which is fitted on the turning shaft 31-1 on the second member 11s2 side. By configuring in this way, the link member 30-1 is fixed by a pin to the second member 11s2. And so the end portion on the movable member 20 side freely swings centered on the turning shaft 31-1.

Also, the link member 30-1 has a shaft hole on the end portion on the movable member 20 side, which is fitted on the turning shaft 32-1 on the movable member 20 side. By configuring in this way, the link member 30-1 is fixed by a pin to the movable member 20. Then, the link member 30-1 is provided such that the end portion on the second member 11s2 side freely swings centered on the turning shaft 32-1.

In the same way, the link member 30-2 has a shaft hole on the end portion of the second member 11s2 side, which is fitted on the turning shaft 31-2 on the second member 11s2 side. By configuring in this way, the link member 30-2 is fixed by a pin to the second member 11s2. Therefore, the link member 30-2 is provided such that the end portion on the movable member 20 side freely swings centered on the turning shaft 31-2.

Also, the link member 30-2 has a shaft hole on the end portion on the movable member 20 side, which is fitted on the turning shaft 32-2 on the movable member 20 side. By configuring in this way, the link member 30-2 is fixed by a pin to the movable member 20. Then, the link member 30-1 is provided such that the end portion on the second member 11s2 side freely swings centered on the turning shaft 32-2.

The four-joint parallel link mechanism that consists of the second member 11s2, the movable member 20, the link member 30-1, and the link member 30-2 is formed by the constitution explained above. Thereby, the movable member 20 is displaced along the longitudinal direction of the grip 11, that is, in the direction shown by the arrow d3 in FIG. 3, accompanying a pressing-down operation of the switch 11s.

Note that in the second member 11s2, on the side surface on the opposite side of the side surface where the aforementioned link members 30-1 and 30-2 are provided, a four-joint parallel link mechanism may similarly be formed. By configuring in this way, it is possible to cause the movable member 20 to undergo parallel displacement more stably.

Also, a well-known link mechanism other than the example described above may be used as a means that converts the displacement according to the downward-push operation of the switch 11s into displacement in the longitudinal direction of the grip 11. Also, the angle relating to the displacement may be arbitrary configured, and a link mechanism may be used such that it becomes an angle in which the displacement direction of the switch 11s and the displacement direction of the movable member 20 are not at a right angle.

The movable member 20 is a bar member that is fixed by a pin to the second member 11s2 with the aforementioned constitution. The movable member 20 reaches the inside of the hollow region 13M of the master arm main body 13, via a through-hole portion 15H of the intermediate member 15 described below. Also, the portion of the movable member 20 on the other side of the portion that faces the switch 11s makes contact with the inner wall surface of the hollow region 11M. A scale 20s for displacement sensing such as an encoder scale or the like is provided at a portion positioned in a hollow region 13M of the master arm main body 13 that is one end of this movable member 20.

The intermediate member 15 is a member whose outer contour has an approximately cylindrical shape that is interposed between the grip 11 and the master arm main body 13 and thereby connects them. This intermediate member 15 is not an essential constituent member. However, since it is possible to cause the grip 11, which belongs to the clean area, to not make direct contact with the master arm main body 13, which belongs to the unclean area, it is possible to further improve the hygiene viewpoint. This intermediate member 15 has an insertion hole portion 15h, a through-hole portion 15H, an engagement groove portion 15d, an insertion projection portion 15i, and an engagement projection portion 15t.

The insertion hole portion 15h is a hole portion in which the insertion projection portion 11i of the grip 11 is inserted to be fitted. The grip 11 and the intermediate member 15 are connected by the fitting of the insertion projection portion 11i and the insertion hole portion 15h.

The through-hole portion 15H is a through hole that is formed along the longitudinal direction of the intermediate member 15. The movable member 20 is inserted into this through-hole portion 15H. The movable member 20 is inserted into the hollow region 13M of the master arm main body 13 via the through-hole portion 15H of the intermediate member 15.

The engagement groove portion 15d is a groove that fits with the engagement projection portion 11t of the grip 11, in the state of the insertion projection portion 11i of the grip 11 being inserted into and fitted with the insertion hole portion 15h of the intermediate member 15. In greater detail, the engagement projection portion 11t and the engagement groove portion 15d engage in the attachment/detachment direction of the grip 11 with respect to the intermediate member 15, to prevent dropping of the grip 11.

The insertion projection portion 15i is a projection portion that is inserted into the insertion hole portion 13h of the mater arm main body 13 described below. The intermediate member 15 and the master arm main body 13 are connected by the insertion projection portion 15i being inserted into the insertion hole portion 13h.

The engagement projection portion 15t is a projection portion that engages with the engagement groove portion 13d of the master arm main body 13 described below.

The drape 17 is a member for separating the “clean area” from the “unclean area” in the master manipulator according to the first embodiment. Here, the “clean area” denotes a region that can be sterilized (a sterilized region). The “unclean area” denotes a region that is difficult to sterilize (an unsterilized region).

Specifically, for example the grip 11 is a part that the operator directly contacts. Assuming a situation arises in which the operator has no other choice but to directly touch a patient's body cavity during a surgical operation, it is necessary to sufficiently perform cleaning and sterilization of the grip 11. Accordingly, the grip 11 should be a “clean area”. Note that for the same reason, the intermediate member 15 that connects the grip 11 and the master arm main body 13 should be a clean area.

On the other hand, since the master arm main body 13 is provided with various electronic components, normally it does not have a structure capable of withstanding a sterilization process. Accordingly, the master arm main body 13 is protected by being wrapped in the drape 17. Also, since mixing of the clean area and the unclean area after sterilization treatment is prevented by separating the clean area from the unclean area by the drape 17, it is possible to improve the hygiene.

The drape connector 19 is an approximately ring shaped member in which a through-hole portion 19h having a diameter larger than the outer diameter of the insertion projection portion 15i of the intermediate member 15 is formed. The drape connector 19 is provided in a portion facing the drape 17 and the intermediate member 15 among the drape 17. The insertion projection portion 15i of the intermediate member 15 is inserted into the insertion hole portion 13h of the master arm main body 13 through the through-hole portion 19h of this drape connector 19.

The master arm main body 13 is provided with a displacement sensor 13s (the position detection portion), an input processing circuit 13c, a hollow region 13M, the insertion hole portion 13h and the engagement groove portion 13d.

The displacement sensor 13s is a displacement sensor such as an incremental encoder or the like. The displacement sensor 13s detects the position or displacement of the movable member 20 by detecting the position of the scale 20s that is provided on the movable member 20.

The input processing circuit 13c calculates the operation amount of the switch 11s, that is, the push-down operation amount, based on the position of the movable member 20 detected by the displacement sensor 13s.

The portion of the one end side of the movable member 20 that is the end at which the scale 20s such as an encoder scale or the like is provided is inserted into the hollow region 13M via the connected intermediate member 15. The displacement sensor 13s for detecting the position or displacement of the movable member 20 is provided at a position corresponding to the movable range of the scale 20s on the inner wall surface of this hollow region 13M.

The position of the movable member 20, which moves in conjunction with the displacement of the switch 11s due to the push-down operation of the switch 11s and the recovery displacement to the original position after the completion of that operation, is detected by the displacement sensor 13s. Then, based on that detection result, the operation amount of the switch 11s is calculated by the input processing circuit 13c.

The insertion hole portion 13h is a hole portion in which the insertion projection portion 15i of the intermediate member 15 is inserted into and fits with. The intermediate member 15 and the master arm main body 13 are connected by the fitting of the insertion projection portion 15i and the insertion hole portion 13h.

The engagement groove portion 13d is a groove portion that engages with the engagement projection portion 15t of the intermediate member 15, in the state of the insertion projection portion 15i of the intermediate member 15 being inserted into and fits with the insertion hole portion 13h of the master arm main body 13. In greater detail, the engagement projection portion 15t and the engagement groove portion 13d engage in the detachment direction of the intermediate member 15 with respect to the master arm main body 13, and prevent the falling out of the intermediate member 15.

As described above, according to the first embodiment, it is possible to provide a master manipulator in which sterilization treatment of the master grip can be easily carried out.

That is to say, no electrical components that are components that may break down by performing sterilization treatment of the grip 11 are arranged in the grip 11 in the master manipulator according to the first embodiment. The master manipulator according to the first embodiment is constituted so as to transmit the operation amount of the switch 11s, which is an operating member that is provided in the grip 11, to the interior of the master arm main body 13 only by a mechanical mechanism that is a component that does not break down even if sterilization treatment is performed.

The displacement sensor 13s and the input processing circuit 13c that are provided in the master arm main body 13 detect the operation amount of the switch 11s. By configuring in this way, it is extremely easy to perform sterilization treatment of the grip 11 in which various operations are possible using the switch 11s.

Hereinabove, the present invention according to the first embodiment was described, but the present invention is not limited to the aforementioned first embodiment, and of course various transformations and applications are possible within the scope of the spirit or scope of the present invention.

### Second Embodiment

Hereinbelow, the master manipulator according to a second embodiment of the present invention shall be described. In order to avoid overlapping of descriptions, the points that differ from the first embodiment shall be described. The main point of difference with the first embodiment is the constitution of the movable member 20 and nearby members.

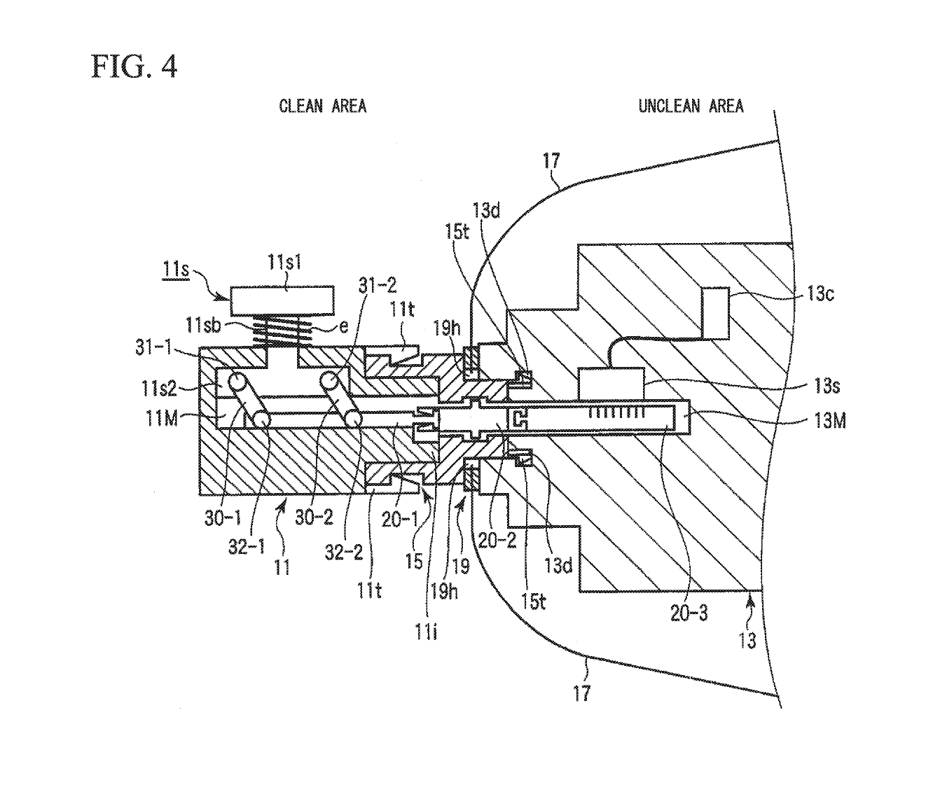

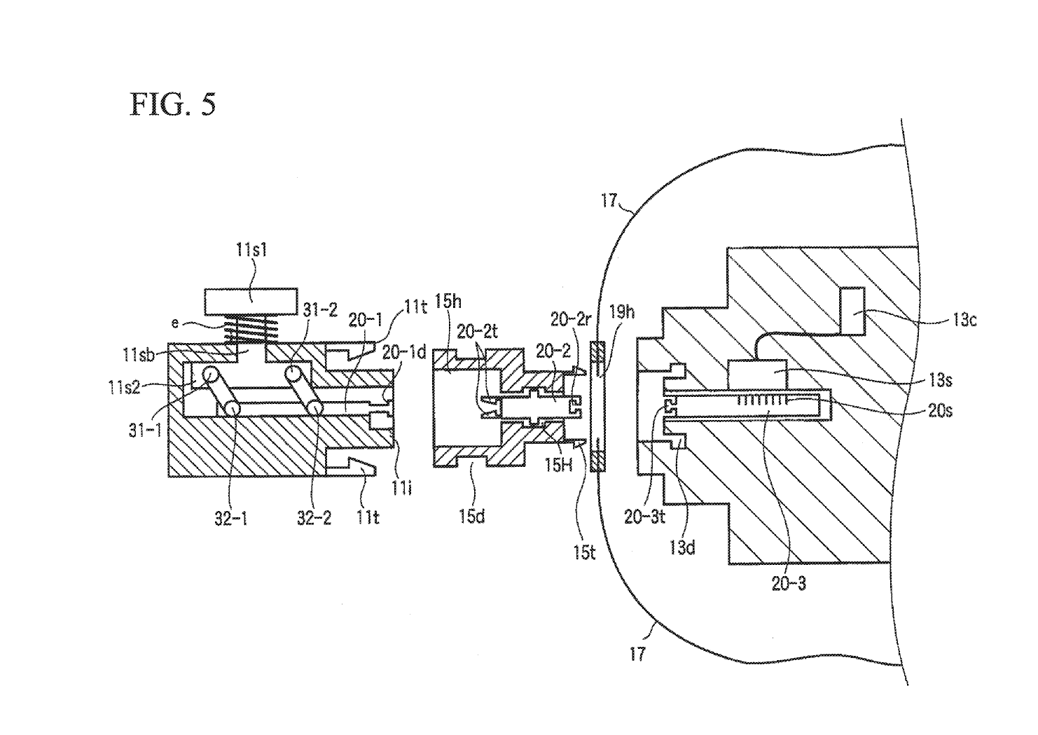

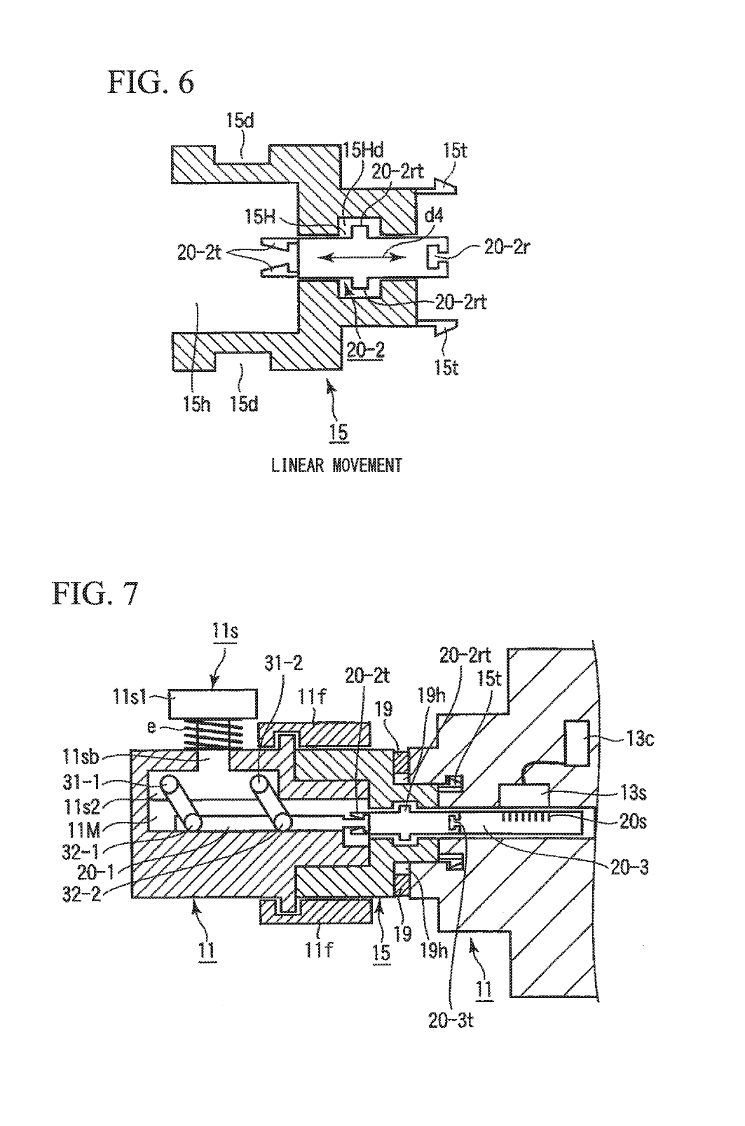

FIG. 4 is a side cross-sectional view that shows one configuration example of the master manipulator according to the second embodiment of the present invention. FIG. 5 is a side cross-sectional view that shows the disassembled state of one configuration example of the master manipulator according to the second embodiment of the present invention. FIG. 6 is a drawing that shows one configuration example of the intermediate member of the master manipulator according to the second embodiment of the present invention.

In the master manipulator according to the second embodiment, the movable member 20 includes a first movable member 20-1 (a first portion) corresponding to the grip 11, a second movable member 20-2 (a second portion) corresponding to the intermediate member 15, and a third movable member 20-3 (a third portion) corresponding to the master arm main body 13.

As shown in FIG. 5, the first movable member 20-1 and the second movable member 20-2 are connected by the engagement of an engagement groove portion 20-1d that is provided in the first movable member 20-1 and an engagement projection portion 20-2t that is provided in the second movable member 20-2.

As shown in FIG. 5, the second movable member 20-2 and the third movable member 20-3 are connected by the engagement of an engagement concave portion 20-2r that is provided in the second movable member 20-2 and an engagement projection portion 20-3t that is provided in the third movable member 20-3.

Here, a scale 20s for sensing by a displacement sensor 13s is provided on the third movable member 20-3 that is housed in the hollow region 13M of the master arm main body 13.

As shown in FIG. 6, a groove portion 15Hd that is formed in the radial direction is provided in the through-hole portion 15H of the intermediate member 15. On the other hand, a regulating projection portion 20-2rt that has projected shape in the radial direction is provided on the second movable member 20-2. The groove portion 15Hd of the intermediate member 15 and the regulating projection portion 20-2rt of the second movable member 20-2 engage. As a result, in the through-hole portion 15H of the intermediate member 15, the advancing and retracting movement of the second movable member 20-2 in the direction shown by the arrow d4 in FIG. 6 is regulated. By having such a configuration, it is possible to set the movement range of the movable member 20 in a desired range. Moreover, during disassembly of the master manipulator, due to the engagement of the groove portion 15Hd of the intermediate member 15 and the regulating projection portion 20-2rt of the second movable member 20-2, it is possible to prevent the second movable member 20-2 from falling out from the intermediate member 15.

The second embodiment as described above exhibits the same effect as the master manipulator according to the first embodiment. Moreover, it is possible to provide a master manipulator with improved usability.

Specifically, by constituting the movable member 20 to be separable at each portion corresponding to the grip 11, the intermediate member 15, and the master arm main body 13, it is possible to more thoroughly separate the clean area from the unclean area in the master manipulator.

### Third Embodiment

Hereinbelow, the master manipulator according to a third embodiment of the present invention shall be described. In order to avoid overlapping of descriptions, the points that differ from the first embodiment shall be described. The main point of difference with the first embodiment is the fixing structure of the grip 11 and the intermediate member 15. In the master manipulator according to the first embodiment, falling out of the grip 11 that is connected to the intermediate member 15 is prevented by the engagement of the engagement projection portion 11t of the grip 11 and the engagement groove portion 15d of the intermediate member 15.

FIG. 7 is a side cross-sectional view that shows one configuration example of the master manipulator according to the third embodiment of the present invention. As shown in FIG. 7, in the master manipulator according to the third embodiment, the grip 11 and the intermediate member 15 are fixed by a fixing member 11f. In the present example, a screw member is used as the fixing member 11f. Note that besides a screw member, a fixing member that utilizes the engagement method of the following so-called BNC connector may be used. That is to say, a structure known as an engagement method of the so-called BNC connector is a structure in which a projection of a male connector is inserted into a hole of a female connector in which an incline is formed, and the male connector and the female connector are connected with a half-rotation twist.

The third embodiment as described above exhibits the same effect as the master manipulator according to the first embodiment. Moreover, it is possible to provide a master manipulator that is capable of more firmly fixing the grip 11 and the intermediate member 15.

### Fourth Embodiment

Hereinbelow, the master manipulator according to a fourth embodiment of the present invention shall be described. In order to avoid overlapping of descriptions, the points that differ from the first embodiment shall be described. One of the main points of difference with the first embodiment is the constitution of the movable member 20 and the constitution for detecting the position or displacement of the movable member 20.

In the master manipulator according to the first embodiment, the scale 20s is provided at a portion of the movable member 20 that is positioned in the hollow region 13M of the master arm main body 13, whereby the position of the scale 20s is detected by a displacement sensor 13s that is provided in the master arm main body 13, and based on that, the operation amount of the switch 11s, that is, the push-down operation amount of the switch 11s, is calculated.

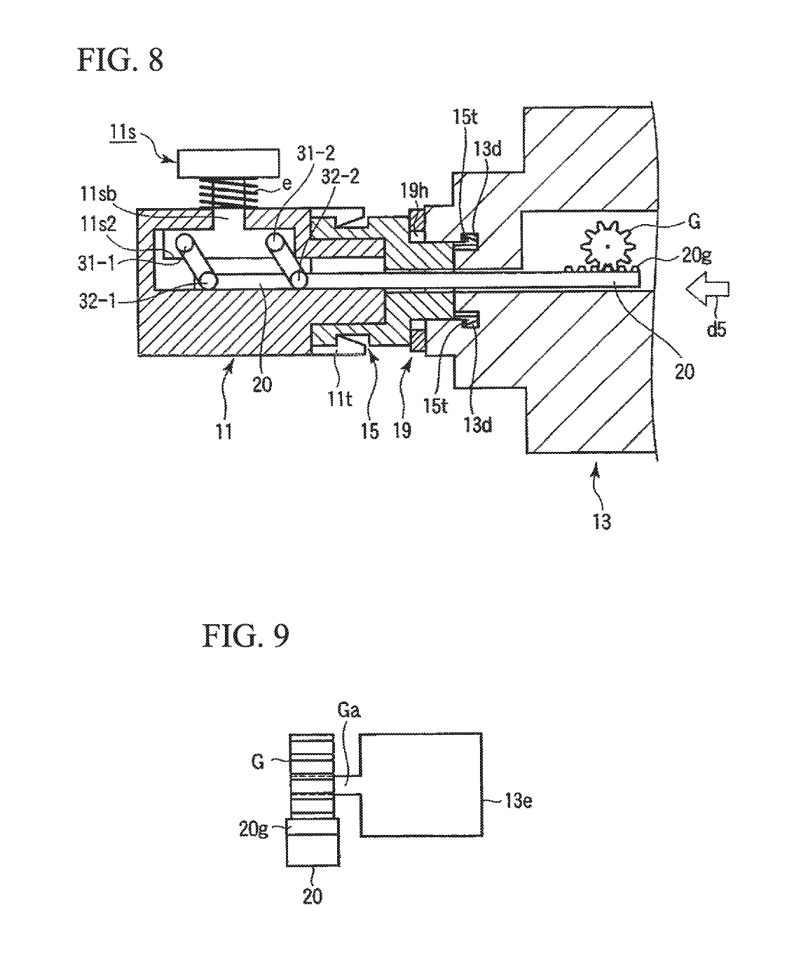

FIG. 8 is a side cross-sectional view that shows one configuration example of the master manipulator according to the fourth embodiment of the present invention. FIG. 9 is a drawing viewing the master manipulator according to the fourth embodiment from the direction shown by the arrow d5 in FIG. 8.

In the master manipulator according to the fourth embodiment, gear cutting is carried out on a portion of the movable member 20 that is positioned in the hollow region 13M of the master arm main body 13, whereby a gear cut portion 20g is formed. Then, a gear wheel G is provided in the hollow region 13M of the master arm main body 13, and forms a so-called rack-and-pinion structure that meshes with the gear cut portion 20g of the movable member 20. The gear wheel G turns in conjunction with the displacement of the movable member 20.

The rotation amount of the gear wheel G is detected by an encoder 13e that is connected to a center shaft Ga of the gear wheel G that is shown in FIG. 9. Based on the rotation amount of the gear wheel G that is detected by this encoder 13e, the position or displacement of the movable member 20, that is to say, the push-down operation amount of the switch 11s, is calculated by an input processing circuit that is not illustrated.

As described above, according to the fourth embodiment, it is possible to provide a master manipulator that exhibits the same effect as the master manipulator according to the first embodiment.

### Fifth Embodiment

Hereinbelow, the master manipulator according to a fifth embodiment of the present invention shall be described. In order to avoid overlapping of descriptions, the points that differ from the first embodiment shall be described. One of the main points of difference with the first embodiment is existence or non-existence of a displacement enlarging mechanism that enlarges the displacement of the movable member 20.

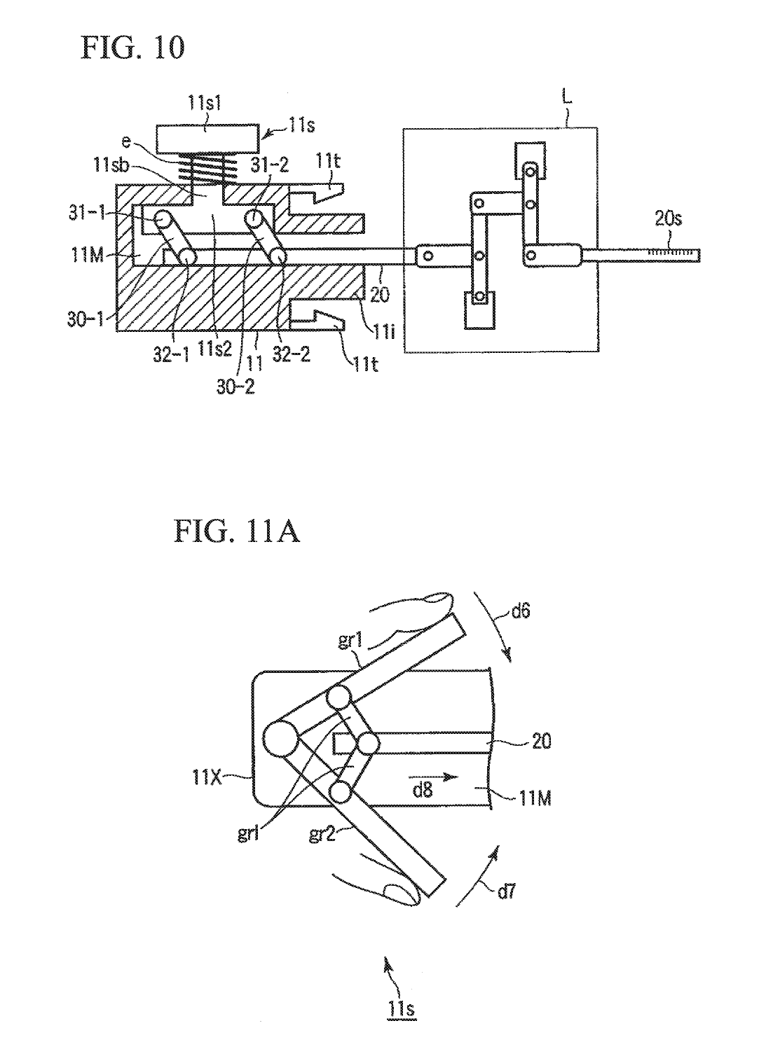

FIG. 10 is a drawing that shows one configuration example of the portion of the displacement enlarging mechanism of the master manipulator according to the fifth embodiment. As shown in FIG. 10, the displacement enlarging mechanism L that is formed by a well-known link mechanism is connected to the movable member 20. In greater detail, the displacement enlarging mechanism L is provided in the space of the movable member 20 from the connecting portion with the switch 11s to the formation portion of the scale 20s. Thereby, the detection ability of the displacement of the movable member 20 improves.

As described above, the fifth embodiment exhibits the same effect as the master manipulator according to the first embodiment. Moreover, according to the fifth embodiment, it is possible to provide a master manipulator in which the operability of the switch 11s is further improved.

### Sixth Embodiment

Hereinbelow, the master manipulator according to a sixth embodiment of the present invention shall be described. In order to avoid overlapping of descriptions, the points that differ from the first embodiment shall be described. One of the main points of difference with the first embodiment is the constitution of the switch 11s that is provided in the grip 11.

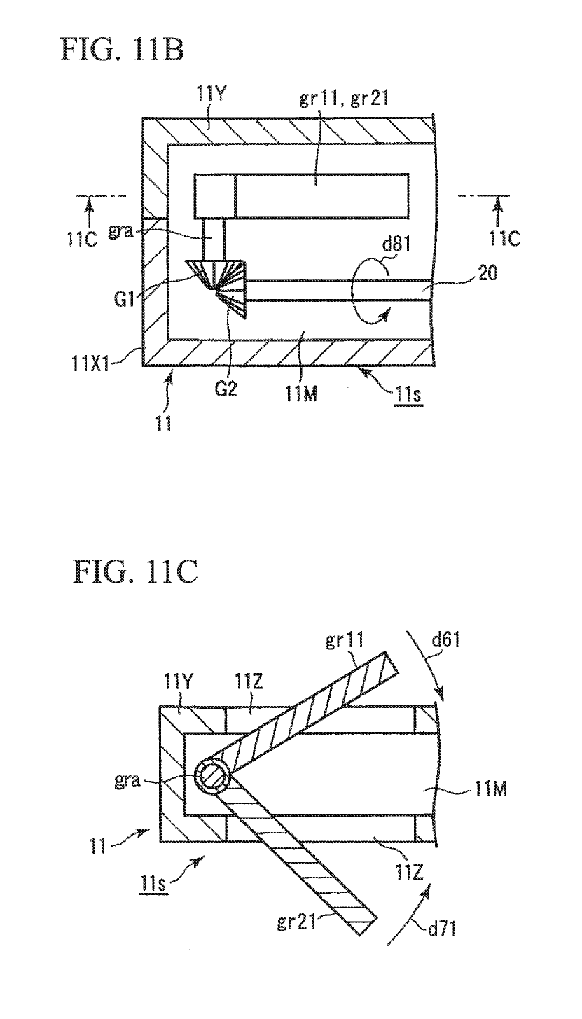

FIG. 11A is a drawing that shows one configuration example of the switch 11s of the master manipulator according to the sixth embodiment. As shown in FIG. 11A, in the master manipulator according to the sixth embodiment, the switch 11s has a first grasping member gr1, a second grasping member gr2, and an opening/closing link gr1 that is connected in a freely opening/closing manner with the first grasping member gr1 and the second grasping member gr2. Note that the grip 11 of the sixth embodiment is provided with a plate-shaped grip main body 11X and a top cover (not illustrated) having a hollow portion that is attached to the grip main body 11X. A cutout is provided in the top cover so that the first grasping member gr1 and the second grasping member gr2 are able to open and close. A hollow region 11M is formed by the grip main body 11X and the top cover. FIG. 11A is a top view of the switch 11s in the state of the top cover having been removed.

In particular, in a medical manipulator, a forceps shape or scissors shape treatment tool is often used by being attached to a slave manipulator. In the case of using a treatment tool that has this kind of opening/closing structure, a well-known opening/closing grip structure is provided in the switch 11s, so that the treatment tool may be opened and closed based on the opening/closing amount related to the opening/closing action of the switch 11s by the fingers of the operator.

In the case of the master manipulator according to the sixth embodiment, the opening/closing amount of the switch 11s by the fingers of the operator, that is to say, the opening/closing amount of the opening/closing grip that is constituted by the first grasping portion gr1 and the second grasping portion gr2, is acquired in the master manipulator, and the treatment tool is opened or closed based on the opening/closing amount.

A constitution that utilizes an opening/closing grip structure by the finger of the operator as an operating member, as in the master manipulator according to the sixth embodiment is not limited to a medical manipulator, and can be applied to all manipulators that use the opening/closing amount due to the opening/closing operation of a switch by the finger of the operator for the operation of the slave manipulator.

Then, by the switch 11s being gripped, external forces are applied in the direction in which the first grasping member gr1 and the second grasping member gr2 mutually approach each other, that is to say, so as to displace the first grasping member gr1 in the direction indicated by the arrow d6, and displace the second grasping member gr2 in the direction indicated by the arrow d7. Then, the movable member 20 displaces in the direction shown by the arrow d8. When the external forces are released, the switch 11s displaces in the opposite direction, that is, displaces to the state prior to the external forces being applied, due to the restitution force that the opening/closing link gr1 has, and in conjunction with this, the movable member 20 also displaces in the opposite direction, that is, displaces to the state prior to the external forces being applied.

That is to say, the switch 11s is constituted so that the movable member 20 advances and retracts in the longitudinal direction, in conjunction with the opening/closing operation of the opening/closing grip that is the operating member. Note that the opening/closing operation of the opening/closing grip in the sixth embodiment is a rotating movement of the first grasping member gr1 and the second grasping member gr2.

As described above, the sixth embodiment can provide a master manipulator that exhibits the same effect as the master manipulator according to the first embodiment.

In addition, in the above example, the movable member 20 is constituted to advance and retract in the longitudinal direction in conjunction with the rotating movement of the first grasping member gr1 and the second grasping member gr2, which are operating members. In addition, it may be constituted so that the movable member 20 turns, and an encoder scale for detecting the rotational displacement of the movable member 20 by the displacement sensor 13s is provided on the side periphery of a portion of the movable member 20 positioned in the hollow region 13M of the master arm main body 13.

FIG. 11B is a side cross-sectional view of the switch 11s of the master manipulator according to the sixth embodiment in the case of that the movable member 20 is constituted to turn. FIG. 11C is a cross-sectional view along 11C-11C in FIG. 11B.

In the configuration example shown in FIG. 11B and FIG. 11C, the switch 11s has a first grasping member gr11, a second grasping member gr21, and a rotating shaft gra. The rotating shaft gra is connected rotatably with the first grasping member gr11 and the second grasping member gr21, and becomes the center of their rotating of the first grasping member gr11 and the second grasping member gr21. Here, gears that constitute “bevel gears” are provided at the distal end of the rotating shaft gra and the distal end of the movable member 20. That is to say, the gear G1 is provided at the distal end of the rotating shaft gra. Also, the gear G2 is provided at the distal end of the movable member 20.

The rotating shaft gra turns by applying an external force so that the first grasping member gr1 and the second grasping member gr2 displace in directions so as to mutually approach. That is to say, by the gripping of the switch 11s, an external force is applied so that the first grasping member gr11 displaces in the direction indicated by the arrow d61, and the second grasping member gr21 displaces in the direction indicated by the arrow d71. At this time, the gear G2, which meshes with the gear G1 that is provided on the rotating shaft gra, rotates, and so the movable member 20 rotationally displaces in the direction indicated by the arrow d81.

Note that the grip 11 according to the sixth embodiment is provided with a grip main body 11X1 that has a bottom and is open at the top and has a hollow portion, and a top cover 11Y that is attached to the grip main body 11X1 and has a hollow portion. A cutout 11Z is provided in the top cover 11Y so that the first grasping member gr11 and the second grasping member gr21 are able to open and close. A hollow region 11M is formed by the grip main body 11X1 and the top cover 11Y.

### Seventh Embodiment

Hereinbelow, the master manipulator according to a seventh embodiment of the present invention shall be described. In order to avoid overlapping of descriptions, the points that differ from the first embodiment shall be described. One of the main points of difference with the first embodiment is the constitution of the switch 11s and the movable member 20.

In the master manipulator according to the first embodiment, the movable member 20 is constituted to advance and retract by a parallel link mechanism that uses link members 30-1 and 30-2. In other words, it is constituted so that, in conjunction with a push-down operation of the switch 11s, the movable member 20 linearly moves to be advanced and retracted displacement. Also, the displacement is detected by the displacement sensor 13s.

On the other hand, the master manipulator according to the seventh embodiment is constituted so that the movable member 20 is rotationally displaced in response to the push-down operation of the switch 11s.

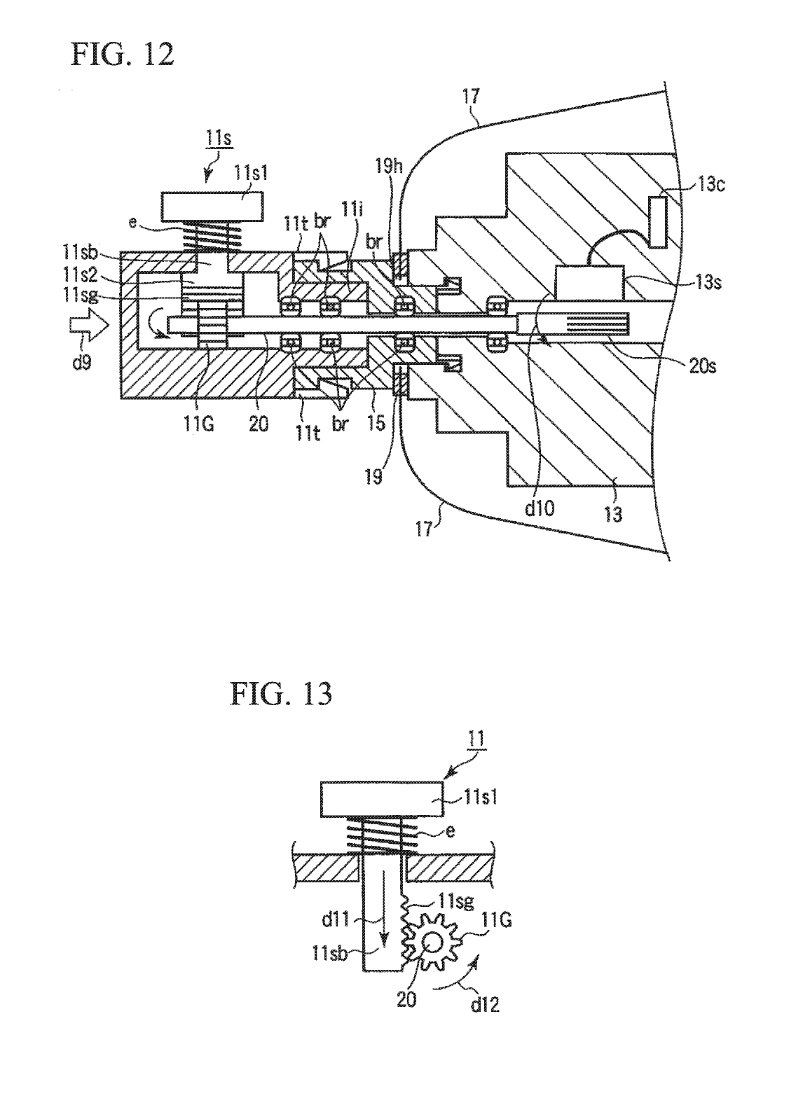

FIG. 12 is a side cross-sectional view that shows one configuration example of the master manipulator according to the seventh embodiment of the present invention. FIG. 13 is a drawing that shows the vicinity of the switch in the case of viewing the master manipulator according to the seventh embodiment of the present invention from the direction indicated by the arrow d9 in FIG. 12.

In the master manipulator according to the seventh embodiment, a gear cut portion 11sg is formed by gear cutting being performed on a side face section of a second member 11s2 of the switch 11s. A gear 11G forms a so-called rack-and-pinion structure by meshing with the gear cut portion 11sg of the second member 11s2, is provided in the movable member 20. In greater detail, the movable member 20 is inserted into the center hole of the gear 11G, and the gear 11G and the movable member 20 are fixed and integrated.

As shown in FIG. 13, the second member 11s2 is displaced in the direction shown by the arrow d11 by the press-down operation of the switch 11s. In conjunction with this, the gear 11G is rotationally displaced in the direction shown by the arrow d12. Then, a bearing member br for facilitating the rotation of the movable member 20 is suitably provided at portions of the grip 11, the intermediate member 15, and the master arm main body 13 that face the movable member 20.

Also, a scale 20s is provided on the side periphery of a portion of the movable member 20 positioned in the hollow region 13M of the master arm main body 13. The scale 20s is an encoder scale for detecting the rotational displacement of the movable member 20 by a displacement sensor 13s. As described above, the seventh embodiment can provide a master manipulator that exhibits the same effect as the master manipulator according to the first embodiment.

### Eighth Embodiment

Hereinbelow, the master manipulator according to an eighth embodiment that is a modified example of the aforementioned seventh embodiment shall be described. In order to avoid overlapping of descriptions, the points that differ from the seventh embodiment shall be described. One of the main points of difference with the seventh embodiment is the constitution of the movable member 20 and members in the vicinity.

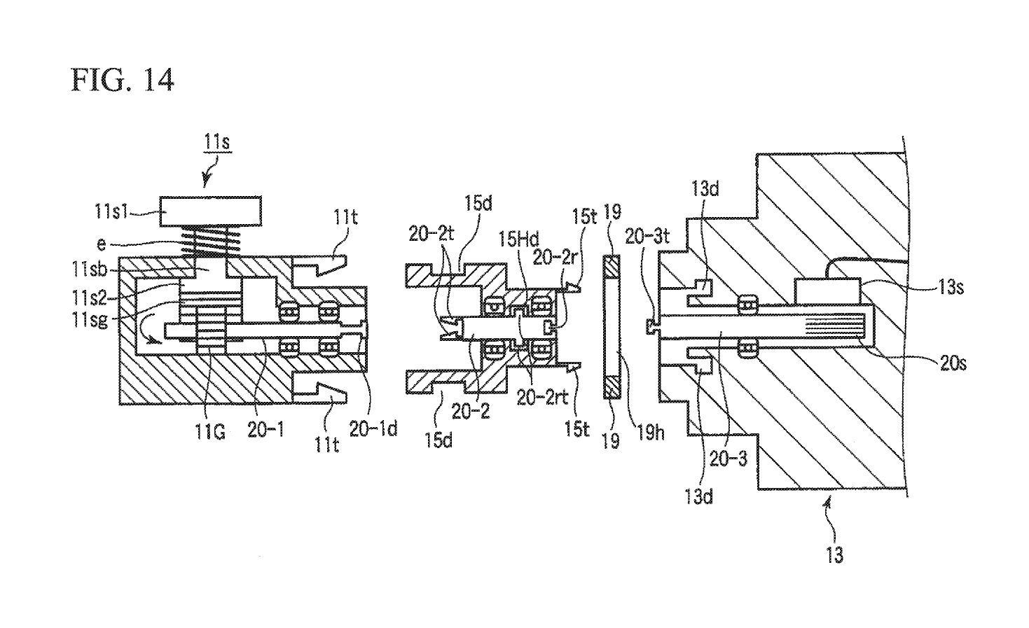

FIG. 14 is a side cross-sectional view that shows one configuration example of the master manipulator according to the eighth embodiment of the present invention.

In the master manipulator according to the eighth embodiment, the movable member 20 includes a first movable member 20-1, a second movable member 20-2, and a third movable member 20-3. Here, the first movable member 20-1 corresponds to the grip 11. The second movable member 20-2 corresponds to the intermediate member 15. The third movable member 20-3 corresponds to the master arm main body 13.

The first movable member 20-1 and the second movable member 20-2 are connected by the engagement of an engagement groove portion 20-1d that is provided in the first movable member 20-1 and an engagement projection portion 20-2t that is provided in the second movable member 20-2.

The second movable member 20-2 and the third movable member 20-3 are connected by the engagement of an engagement concave portion 20-2r that is provided in the second movable member 20-2 and an engagement projection portion 20-3t that is provided in the third movable member 20-3.

Here, a scale 20s for sensing by a displacement sensor 13s is provided on the third movable member 20-3 that is housed in the hollow region 13M of the master arm main body 13.

A groove portion 15Hd that is formed in the radial direction is provided in the through-hole portion 15H of the intermediate member 15. On the other hand, a regulating projection portion 20-2rt that projects in the radial direction is provided on the second movable member 20-2. By the engagement of the groove portion 15Hd of the intermediate member 15 and the regulating projection portion 20-2rt of the second movable member 20-2, the movement in the longitudinal direction of the second movable member 20-2 in the through-hole portion 15H of the intermediate member 15 is regulated. By having such a configuration, it is possible to inhibit wobbling of the movable member 20 in the longitudinal direction.

The eighth embodiment as described above exhibits the same effect as the master manipulator according to the seventh embodiment. Moreover, the eighth embodiment can provide a master manipulator with improved usability.

Specifically, by constituting the movable member 20 to be separable at each portion corresponding to the grip 11, the intermediate member 15, and the master arm main body 13, it is possible to more thoroughly separate the clean area from the unclean area in the master manipulator.

### Ninth Embodiment

Hereinbelow, the master manipulator according to a ninth embodiment that is a modified example of the aforementioned seventh embodiment shall be described. In order to avoid overlapping of descriptions, the points that differ from the seventh embodiment shall be described. One of the main points of difference between the ninth embodiment and the seventh embodiment is the existence or non-existence of a displacement enlargement mechanism that enlarges the displacement of the movable member 20.

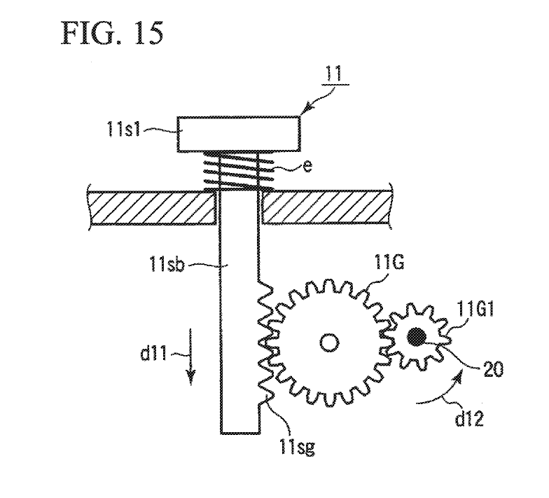

FIG. 15 is a drawing that shows the vicinity of the switch 11s of the grip 11 of the master manipulator according to the ninth embodiment of the present invention, viewed from the direction indicated by the arrow d9 in FIG. 12.

As shown in FIG. 15, a gear 11G forms a so-called rack-and-pinion structure together with the switch 11s that has a gear cut portion 11sg on the second member 11s2. A gear 11G1 with a smaller diameter than the gear 11G is provided meshed with this gear 11G. Then, the movable member 20 is inserted into a center hole of this gear 11G1 to be fixed to the gear 11G1. By configuring in this manner, the detectability of the displacement of the movable member 20 is improved.

As described above, the ninth embodiment exhibits the same effect as the master manipulator according to the seventh embodiment. Moreover, the ninth embodiment can provide a master manipulator with further improved operability of the switch 11s.

### Tenth Embodiment

Hereinbelow, the master manipulator according to a tenth embodiment that is a modified example of the aforementioned seventh embodiment shall be described. In order to avoid overlapping of descriptions, the points that differ from the seventh embodiment shall be described. One of the main points of difference with the seventh embodiment is the constitution for rotationally displacing the movable member 20 in conjunction with a push-down operation of the switch 11s.

In the master manipulator according to the aforementioned seventh embodiment, the movable member 20 is made to perform turned movement by linear movement of the switch 11s, using a rack-and-pinion structure. However, a well-known link mechanism may be used to convert the linear movement to rotational movement.

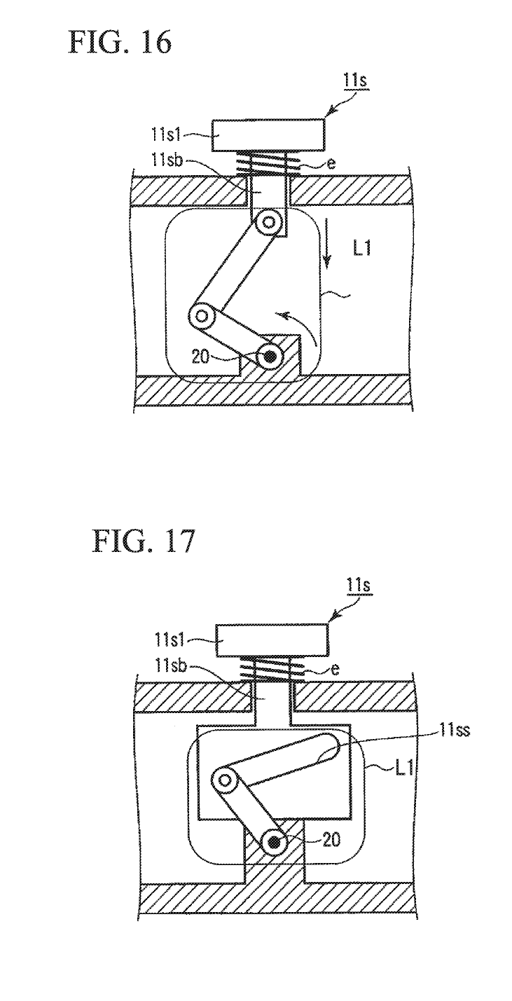

FIG. 16 and FIG. 17 are drawings that show one configuration example of the vicinity of the switch 11s of the grip 11 of the master manipulator according to the tenth embodiment of the present invention, and are drawings viewing the master manipulator from the direction indicated by the arrow d9 in FIG. 12. As shown in FIG. 16 and FIG. 17, the switch 11s and the movable member 20 are connected by a displacement enlargement mechanism L1. The displacement enlargement mechanism L1 is a well-known link mechanism for converting linear movement to turning movement. The displacement enlargement mechanism L1 shown in FIG. 17 is a displacement enlargement mechanism that utilizes a link mechanism with a configuration that has a slide groove 11ss.

Note that the mechanism for causing the movable member 20 to perform turning movement by linear movement of the switch 11s is not limited to the example shown in FIG. 16 and FIG. 17. Provided it is a mechanism that is capable of causing the movable member 20 to perform turning movement by linear movement of the switch 11s, any kind of mechanism may be used.

As described above, the tenth embodiment can provide a master manipulator that exhibits the same effect as the master manipulator according to the seventh embodiment.

### Eleventh Embodiment

Hereinbelow, the master manipulator according to an eleventh embodiment that is a modified example of the aforementioned first embodiment shall be described. In order to avoid overlapping of descriptions, the points that differ from the first embodiment shall be described. One of the main points of difference with the first embodiment is the constitution for causing the movable member 20 to linearly move to perform advancing/retracting linear displacement in conjunction with the operation of the switch 11s.

In the master manipulator according to the first embodiment, the movable member 20 is constituted to be capable of advancing and retracting by constituting a parallel link mechanism that uses link members 30-1 and 30-2. In other words, the movable member 20 is constituted to linearly move to perform advancing/retracting displacement in conjunction with the push-down operation of the switch 11s. And then, this displacement is detected by the displacement sensor 13s. On the other hand, in the master manipulator according to the eleventh embodiment, the movable member 20 is made to linearly move to perform advancing/retracting displacement in conjunction with the rotational operation of the switch 11s.

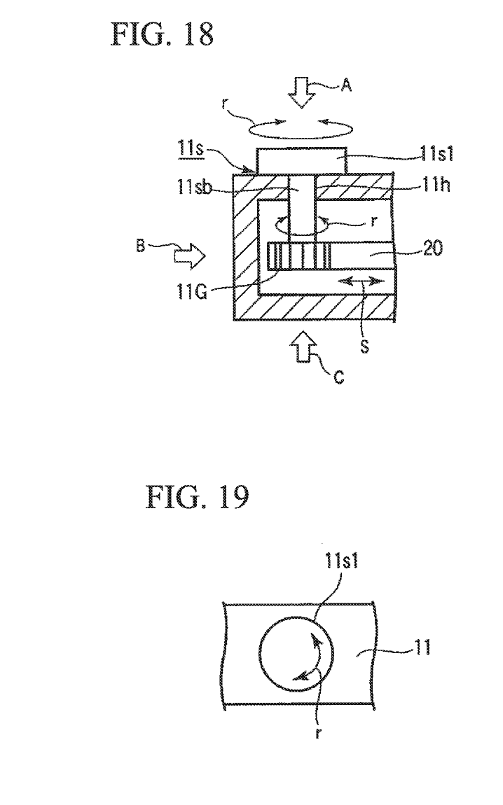

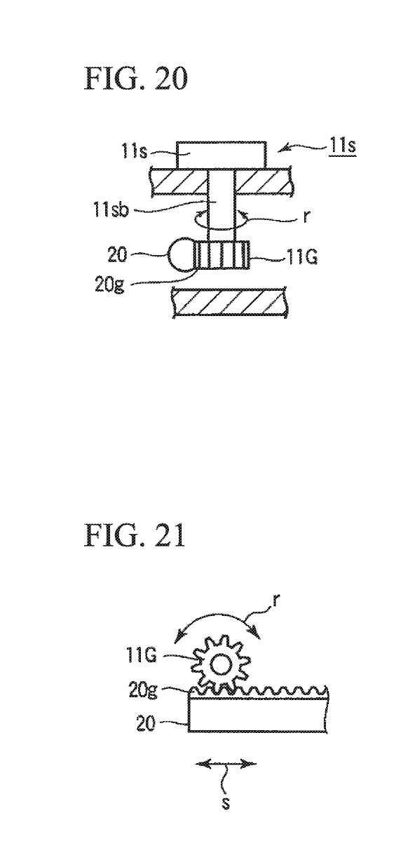

FIG. 18 is a side cross-sectional view that shows the vicinity of the switch of the grip of the master manipulator according to the eleventh embodiment. FIG. 19 is a top view of the switch vicinity of the grip of the master manipulator according to the eleventh embodiment, viewed from the direction shown by the arrow A in FIG. 18. FIG. 20 is a view of the switch vicinity of the grip of the master manipulator according to the eleventh embodiment, viewed from the direction indicated by the arrow B in FIG. 18. FIG. 21 is a view of the switch vicinity of the grip of the master manipulator according to the eleventh embodiment, viewed from the direction indicated by the arrow C in FIG. 18.

As shown in FIG. 18 to FIG. 21, the switch 11s includes a first member 11s1, a bar member 11sb, and a gear 11G. The first member 11s1 is rotationally operated by an operator in a direction indicated by the arrow r. The bar member 11sb is inserted into a through hole 11h of the grip 11, with one end fixed to the first member 11s1. The gear 11G is provided at the other end of the bar member 11sb.

Gear cutting is performed at a section of the movable member 20 positioned at the hollow region 11M of the grip 11, whereby a gear cut portion 20g is formed. Then, a so-called rack-and-pinion structure is formed by this gear cut portion 20g meshing with the gear 11G.

By adopting the aforementioned configuration, when the first member 11s1 of the switch 11s is rotationally operated in the direction indicated by the arrow r, the gear cut portion 20g that meshes with the gear 11G is sent out by the gear 11G. As a result, the movable member 20 linearly moves in the direction shown by the arrow s, and perform advancing/retracting displacement.

That is to say, in the master manipulator according to the eleventh embodiment, by utilizing a rack-and-pinion structure, the movable member 20 is made to linearly move to perform advancing/retracting displacement by the rotational operation of the switch 11s.

As described above, the eleventh embodiment can provide a master manipulator that exhibits the same effect as the master manipulator according to the first embodiment.

### Twelfth Embodiment

Hereinbelow, the master manipulator according to a twelfth embodiment shall be described. In order to avoid overlapping of descriptions, the points that differ from the first embodiment shall be described. One of the main points of difference with the first embodiment is the constitution of the displacement sensor 13s.

In the master manipulator according to the first embodiment, the movable member 20 is constituted to linearly move to perform advancing/retracting displacement or to perform rotational displacement in the master arm main body 13 in conjunction with a push-down operation of the switch 11s. The displacement is detected by the displacement sensor 13s.

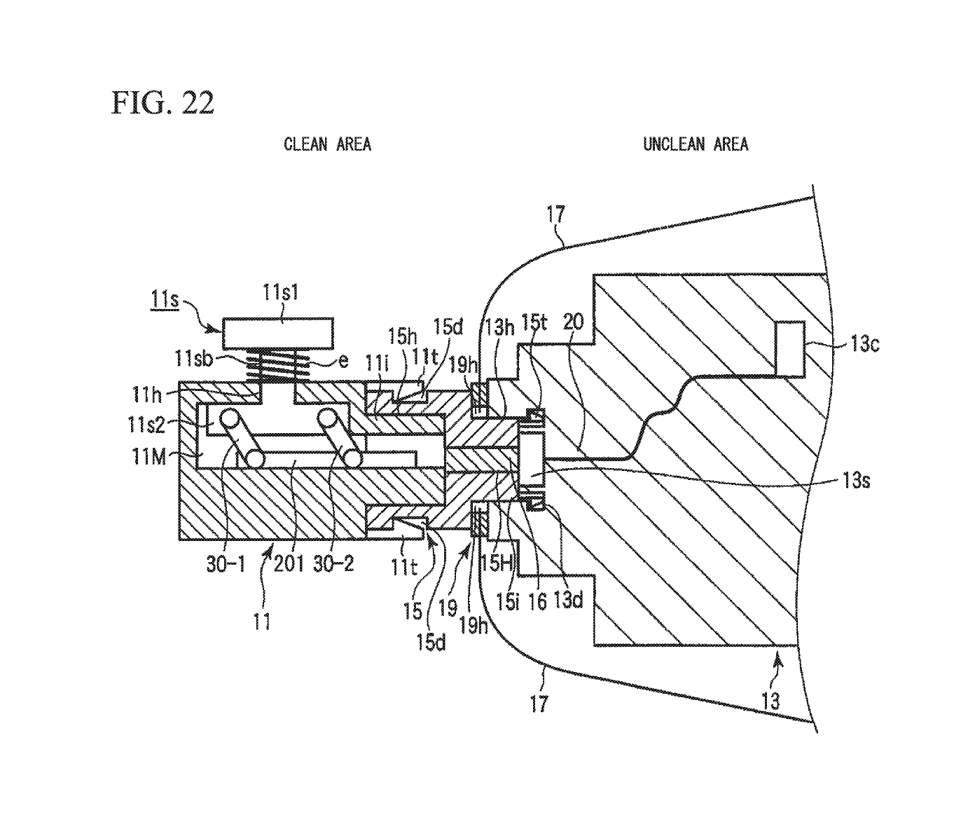

On the other hand, in the master manipulator according to the twelfth embodiment, as shown in FIG. 22, a movable member 201 advances and retracts only in the hollow region 11M that is in the grip 11 in conjunction with a push-down operation of the switch 11s. The displacement of the movable member 201 in the hollow region 11M of the interior of the grip 11 is acquired by the displacement sensor 13s in the interior of the master arm main body 13.

Note that the master manipulator according to the present embodiment is not one that is limited to the configuration example shown in FIG. 22. In addition, although not illustrated, the switch 11s may be constituted to perform a rotating operation or a turning operation, and the movable member 201 may be constituted so as to turn within the hollow region 11M in conjunction with that rotating operation or turning operation.

FIG. 22 is a side cross-sectional view that shows one configuration example of the master manipulator according to the twelfth embodiment of the present invention.

As shown in FIG. 22, in the master manipulator according to the twelfth embodiment, the movable member 201 has a length such that the movable member 201 is able to advance or retract only within the hollow region 11M that is in the grip 11 by the push-down operation of the switch 11s. The displacement sensor 13s is provided at a position where the movable member 201 faces the end face of the master arm main body 13 side.

The displacement sensor 13s is installed so that the reception face of the displacement sensor 13s matches the connection face of the master arm main body 13 and the intermediate member 15. Moreover, a transmission member 16 that is made of a transparent resin or the like is provided at the center portion of the intermediate member 15 so as not to impede the reception by the displacement sensor 13s. While the transmission member 16 is not essential, by having this kind of constitution, it is possible to completely separate the clean area from the unclean area, and so the sterilization property improves.

Specifically, as the displacement sensor 13s, it is possible to use various displacement sensors that are well-known, such as laser light, infrared light, ultrasonic waves, magnetism or the like. In order to accurately detect the displacement of the moving sensor 201 by the displacement sensor 13s, it is preferable that a material that transmits the light, ultrasonic waves, magnetism or the like used by the displacement sensor 13s be made the material of the transmission member 16.

As the method of detecting the position of the movable member 201 using this displacement sensor 13s, there is for example the following method.

That is to say, in the case of the transmission member 16 being in the path from the displacement sensor 13s to the movable member 201, a laser light, infrared light, ultrasonic, or magnetic signal is emitted to the movable member 201 via the transmission member 16. Then, the signal that is reflected from the movable member 201 is received by the displacement sensor 13s via the transmission member 16, whereby the position or displacement of the movable member 201 is detected.

Note that the master manipulator according to the present embodiment is not limited to the arrangement of the displacement sensor 13s and the movable member 201 in the example shown in FIG. 22 described above. For example, the displacement of the movable member 201 may also be detected by arranging a reflecting member such as a mirror or the like to reflect or refract a laser light, infrared light, ultrasonic, or magnetic signal between the displacement sensor 13s and the movable member 201, and then detecting the displacement of the movable member 201 by detecting that signal by the displacement sensor 13s.

By adopting such a constitution, the movable member 201 and the displacement sensor 13s need not be arranged so as to face each other, and so degree of freedom of design increases.

Also, the section of the movable member 201 that reflects the signal from the displacement sensor 13s, for example the end face on the side of the master arm main body 13 (displacement sensor 13s), is preferably constituted by a material that reflects the signal, or a reflecting member is preferably provided at that end face. With this constitution, it is possible to accurately detect the displacement of the movable member 201 by the displacement sensor 13s.

Displacement detection systems include a system of reading a scale attached to the movable member as in the first embodiment, and a system that utilizes the time differential of the signal that is received by the displacement sensor 13s.

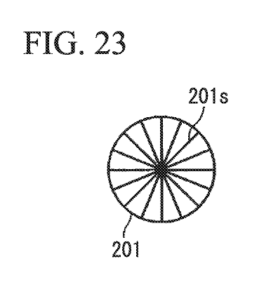

Note that although not illustrated, it is possible to measure the displacement by the arrangement of the same displacement sensor 13s in the case of the movable member 20 rotating as in the aforementioned seventh embodiment shown in FIG. 12. In the case of being constituted in this way, the way of cutting the scale 20s, that is to say, the way of performing the pattern formation of the scale 20, differs from the first embodiment. Here, FIG. 23 is a front view that shows a scale 201s of the movable member 201 of the master manipulator according to the twelfth embodiment. That is to say, the scale 201s should be formed in a radial fashion in the radial direction from the center axis of the movable member 201 to the outer periphery, as shown for example in FIG. 23, at the end face on the side of the master arm main body 13 of the movable member 201.

Moreover, inventions of various stages are included in each embodiment, and various inventions can be extracted by appropriately combining the plurality of essential components disclosed therein. For example, even when some constituent elements are deleted from all the essential components disclosed in each embodiment, provided the problem to be solved given above is solvable and the effect given above is attainable, then, a configuration from which these essential components have been deleted can be extracted as an invention. Accordingly, the invention is not to be considered as limited by the foregoing description and is only limited by the scope of the appended claims.

What is claimed is:

1. A master manipulator for operating a driving of a slave manipulator, the master manipulator comprising:
a master arm main body;
an intermediate member detachably engaged with the master arm main body;
a grip portion detachably engaged with the intermediate member and disposed at a position which is separated from the master arm main body, the grip portion being configured to be gripped by an operator;
an operating member disposed so as to be exposed from an outer surface of the grip portion and configured to be operated by the operator; and
a movable member including a first portion, a second portion, and a third portion, the first portion being configured to move inside the grip member in accordance with an operation of the operating member by the operator, the second portion being detachably engaged with the first portion and being configured to move inside the intermediate member in accordance with a movement of the first portion, the third portion being detachably engaged with the second portion and being configured to move inside of the master arm main body in accordance with a movement of the second portion,
wherein in a state where the master arm main body, the intermediate member, and the grip portion are detached, the first portion positioned inside the grip member, the second portion positioned inside the master arm main body, and the third portion positioned inside the master arm main body are detached.

2. The master manipulator according to claim 1,
wherein the intermediate member is capable of engaging with the master arm main body so as to hold a drape.

3. The master manipulator according to claim 2,
wherein the intermediate member is capable of engaging with the master arm main body such that the drape is held sandwiched between the master arm main body and the intermediate member.

4. The master manipulator according to claim 1,
wherein the first portion is engaged with the second portion such that the first portion is capable of detaching the second portion in a moving direction of the first portion in a state where the master arm main body and the intermediate member are detached, and
the second portion is engaged with the third portion such that the second portion is capable of detaching the third portion in a moving direction of the second portion in a state where the intermediate portion and the grip portion are detached.

5. The master manipulator according to claim 4,
wherein in a state that the master arm main body, the intermediate member, and the grip portion are engaged with each other, the first portion, the second portion, and the third portion are engaged with each other so as to be capable of transferring force for moving in axial directions thereof in accordance with an operation of the operating member by the operator.

6. The master manipulator according to claim 1, further comprising a transmission member disposed between the grip portion and the master arm main body,
wherein the grip portion is positioned at a clean area and a hollow space is formed in the grip portion,
the master arm main body is positioned at an unclean area and the grip portion is directly or indirectly connected with the master arm main body,
the movable member moves inside the hollow space in conjunction with displacement of the operating member, and
the master arm main body has a position detection portion detecting a position of the movable member inside the hollow space via the transmission member.

7. The master manipulator according to claim 6,
wherein the movable member is a member that advances and retracts in a longitudinal direction in conjunction with a linear movement of the operating member; and
the position detection portion detects linear displacement of the movable member.

8. The master manipulator according to claim 6,
wherein the movable member is a shaft member that rotates in conjunction with a linear movement of the operating member; and
the position detection portion detects rotational displacement of the movable member.

9. The master manipulator according to claim 6,
wherein the movable member is a member that advances and retracts in a longitudinal direction in conjunction with a rotating or turning movement of the operating member; and
the position detection portion detects an linear displacement of the movable member.

10. The master manipulator according to claim 6,
wherein the movable member is a shaft member that rotates in conjunction with a rotating or turning movement of the operating member; and
the position detection portion detects tuning displacement of the movable member.

11. The master manipulator according to claim 6, further comprising a displacement enlargement mechanism that enlarges and transmits the displacement of the operating member,
wherein the position detection portion detects the position of the movable member that moves in conjunction with the displacement that is enlarged and transmitted by the displacement enlargement mechanism.
